(19) United States
(12) Patent Application Publication
(10) Pub. No.: US 2024/0024240 A1
(43) Pub. Date: Jan. 25, 2024

(54) A NANOPARTICLE MATERIAL, A PROCESS FOR OBTAINING THE NANOPARTICLE MATERIAL, A PRODUCT COMPRISING THE NANOPARTICLE MATERIAL AND USES THEREOF

(71) Applicants: B. Braun Surgical S.A., Rubi (ES); Universitat Politècnica de Catalunya, Barcelona (ES); Fundació Institut de Investigació en Ciències de la Salut Germa Trias i Pujol, Badalona (ES); Fundació Institut de Recerca contra la Leucemia Josep Carreras, Barcelona (ES); Institució Catalana de Recerca i Estudis Avançats, Barcelona (ES)

(72) Inventors: Pau Turón Dols, Rubí (Barcelona) (ES); Christine Weis, Rubí (Barcelona) (ES); Vanesa Sanz Beltrán, Rubí (Barcelona) (ES); Carlos Enrique Alemán Llansó, Barcelona (ES); Juan Torras Costa, Barcelona (ES); Juan Francisco Julián Ibáñez, Badalona (ES); Carolina De la Torre, Barcelona (ES); Manel Esteller Badosa, Barcelona (ES)

(21) Appl. No.: 17/918,957

(22) PCT Filed: Apr. 14, 2021
(86) PCT No.: PCT/EP2021/059687

§ 371 Date: Oct. 14, 2022

(30) Foreign Application Priority Data
Apr. 14, 2020 (EP) 20382293.7

(51) Int. Cl. A61K 9/14; A61K 51/12

(52) U.S. Cl. CPC A61K 9/143; A61K 51/1244; A61K 2121/00; B82Y 5/00

(57) ABSTRACT

A nanoparticle material, a process for obtaining the nanoparticle material, a product that includes the nanoparticle material, and methods of using the nanoparticle material. The nanoparticle material includes a resorbable nanoparticle that is functionalized with at least one compound.

### CROSS-REFERENCE TO RELATED APPLICATIONS

This application is the United States national stage entry of International Application No. PCT/EP2021/059687, filed Apr. 14, 2021, and claims priority to European Application No. 20 382 293.7, filed Apr. 14, 2020. The contents of International Application No. PCT/EP2021/059687 and European Application No. 20 382 293.7 are incorporated by reference herein in their entireties.

### FIELD

The present invention relates to a nanoparticle material, a process for obtaining the nanoparticle material, a product, in particular a pharmaceutical composition, a medical kit or a medical device, comprising the nanoparticle material and to various uses of the nanoparticle material.

### BACKGROUND

New strategies for treating current infections by pathogens from viral, bacterial or viral origin, for example a viral infection, such as the last outbreak caused by SARS-CoV-2, require: a) innovative approaches to inactivate the pathogen in physiological environments, and b) new techniques to disinfect contaminated air and surfaces, particularly medical devices intended to protect health care professionals. The first are required to minimize the infection rate and to reduce or palliate the effects of the pathogen infection and their consequences as pneumonia in the case of SARS-CoV-2 and the most critical cases, the death of the patient. The second are intended to guarantee the reduction or inactivation of the pathogen in contaminated environments able to reduce the pathogen already spread in aerosols or remaining in surfaces in order to obtain enhanced protection of patients and health care professionals that need to use protective medical devices (i.e. masks or gloves) and the extension of their use and recyclability.

Classical procedures to obtain an effective pharmacological treatment to fight pathogens, in particular virus, are tedious and relatively slow compared to the immediateness of the outbreak. For example, it is necessary to develop novel strategies to speed-up the SARS-CoV-2 infection treatment.

Nanotechnology can be a successful approach against several pathogens. Nanotechnology involves the use of particulate materials which have at least one dimension of less than 100 nanometers in length. In last years, the field of nanotechnology has developed an increasing amount of applications in different areas, particularly interesting are those focused on medicine. Such a research field remains scarcely unexplored and the development of safe and efficient nanodevices for both disinfection and therapy still remains a significant challenge.

On the other hand, nanotechnology encounters some concerns due to its potentially harmful secondary effects, in terms of toxicity and biocompatibility. With respect to therapeutic fields of use the removal of nanomaterials from the organism after the treatment represents a major aspect. Studies about the toxicity, cytotoxicity and biodistribution of nanomaterials have been performed showing that many types of nanoparticles are biocompatible and neither toxic nor cytotoxic but have confirmed that nanoparticles are accumulated in certain tissues and organs. The development of a strategy for the clearance of these accumulated nanoparticles from the body is still an incompletely explored field and remains as a main challenge.

The functionalization of nanoparticles to capture virus is a novel field or research. Few studies have been focused on such an indication. It is worth mentioning that the impact of a gold nanoparticles on virus was described reporting that non-toxic nanoparticles might eventually lead to irreversible viral deformation and further inactivation (V. Cagno, P. Andreozzi, M. D′Alicarnasso6, P. J. Silva, M. Mueller, M. Galloux, R. Le Go-c7, S. T. Jones, M. Vallino, J. Hodek, J. Weber, S. Sen, E.-R. Janecek, A. Bekdemir, B. Sanavio, C. Martinelli, M. Donalisio, M.-A. R Welti, J.-F. Eleouet, Y. Han, L. Kaiser, L. Vukovic, C. Tapparel, P. Kral, S. Krol, D. Lembo, F. Stellacci, Broad-spectrum non-toxic antiviral nanoparticles with a virucidal inhibition mechanism, Nature Materials, 2017, 17, 195-204).

It is essential to establish the basis for the virus inactivation and to perform the toxicological risk assessment before authorization of any application that includes nanoparticles. The current nanoparticles used in biomedicine include the usage of metallic ions such as gold, silver and zinc, however, those nanoparticles trigger significant concerns regarding their toxicity due to the risks associated with heavy metal elements and their accumulation in the body.

Accordingly, there is still a need of novel therapeutic strategies to fight against pathogens, such as virus, or even against a proliferative disease such as cancer.

### SUMMARY

In view of the foregoing, the object underlying the present invention is therefore to make available a solution based on nanotechnology that is applicable both to non-medical fields of use and medical fields of use, preferably medical fields of use, in particular for preventing and/or treating diseases and/or disorders, that at least partially avoids the above-mentioned disadvantages in the context of conventional nanotechnology.

According to a first aspect the present invention relates to a nanoparticle material comprising a resorbable nanoparticle being functionalized with at least one compound or consisting of a resorbable nanoparticle being functionalized with at least one compound. In other words, according to a first aspect, the present invention relates to a nanoparticle material comprising or consisting of a resorbable functionalized nanoparticle, wherein the resorbable nanoparticle is functionalized with at least one compound.

Within the scope of the present invention the nanoparticle material may also be termed as “resorbable functionalized nanoparticle”, in particular “selective resorbable functionalized nanoparticle”.

The term “functionalized” in the context of the resorbable nanoparticle as used according to the present invention means to endow or equip the resorbable nanoparticle with at least one functionality, which the resorbable nanoparticle normally, i.e. in an unfunctionalized condition, does not possess, by reacting the resorbable nanoparticle with at least one compound resulting in an attachment, in particular a covalent and/or non-covalent attachment, of the at least one compound or a moiety thereof to the resorbable nanoparticle or a surface thereof. Preferably, the functionality may be or comprise at least one functional group. More preferably, the at least one functional group has an affinity, in particular in terms of binding, in particular binding strength, and/or selectivity, to the resorbable nanoparticle and/or the surface thereof. In particular, the functionality may be in the form of a chelating group comprising at least one functional group. For example, the at least one functional group may be selected from the group consisting of carboxyl group, hydroxyl group, amine group, phosphate group, phosphonate group, bisphosphonate group, sulphonate group and combinations of at least two of the afore-mentioned functional groups.

The term “nanoparticle” as used according to the present invention refers to a particle or particulate material having at least one dimension, in particular a diameter, preferably an average diameter, and/or a length, preferably an average length, and/or a width, in particular an average width, and/or a height, in particular an average height, of ≤500 nm, in particular from 0.1 nm to ≤500 nm, in particular 0.1 nm to ≤250 nm, preferably 0.1 nm to ≤100 nm, more preferably 1 nm to ≤100 nm, in particular 1 nm to 100 nm or 1 nm to <100 nm, preferably 10 nm to 50 nm, in particular including any integer and/or decimal number included in the afore-mentioned ranges. The at least one dimension, in particular the diameter, preferably average diameter, and/or length, preferably average length, and/or width, in particular average width, and/or height, in particular average height, may be in particular determined by means of Transmission Electron Microscopy (TEM).

Thus, the term “nanoparticle” as used according to the present invention may mean a nanometric particle, i.e. a particle having at least one dimension, in particular a diameter, preferably an average diameter, and/or a length, preferably an average length, and/or a width, in particular an average width, and/or a height, in particular an average height, of ≤100 nm, preferably from 0.1 nm to ≤100 nm, more preferably 1 nm to ≤100 nm, in particular 1 nm to 100 nm or 1 nm to <100 nm, preferably 10 nm to 50 nm, and/or a submicrometric particle, i.e. a particle having at least one dimension, in particular a diameter, preferably an average diameter, and/or a length, preferably an average length, and/or a width, in particular an average width, and/or a height, in particular an average height, from >100 nm to ≤500 nm, in particular >100 nm to 500 nm or >100 nm to <500 nm.

Accordingly, the term “resorbable nanoparticle” as used according to the present invention may also be termed as “resorbable nanometric particle and/or resorbable submicrometric particle”. Further, the term “nanoparticle material” as used according to the present invention may also be termed as “nanometric material and/or submicrometric material”.

Preferably, the term “nanoparticle” as used according to the present invention means a nanometric particle, i.e. a particle having at least one dimension, in particular a diameter, preferably an average diameter, and/or a length, preferably an average length, and/or a width, in particular an average width, and/or a height, in particular an average height, of ≤100 nm, preferably from 0.1 nm to ≤100 nm, more preferably 1 nm to ≤100 nm, in particular 1 nm to 100 nm or 1 nm to <100 nm, preferably 10 nm to 50 nm (a so-called nanoparticle in the strict sense of the word).

The term “a resorbable nanoparticle” as used according to the present invention may mean only one resorbable nanoparticle or a plurality of resorbable nanoparticles. In the latter case, the resorbable nanoparticles may be equal or different.

The term “a resorbable nanometric particle” as used according to the present invention may mean only one resorbable nanometric particle or a plurality of resorbable nanometric particles. In the latter case, the resorbable nanometric particles may be equal or different.

The term “a resorbable submicrometric particle” as used according to the present invention may mean only one resorbable submicrometric particle or a plurality of resorbable submicrometric particles. In the latter case, the resorbable submicrometric particles may be equal or different.

The term “selective resorbable functionalized nanoparticle” as used according to the present invention means a nanoparticle being functionalized with at least one compound, in particular at least one ligand, that is able to selectively bind to a pathogen and/or to a proliferative cell, in particular tumoral cell.

In general, the use of nanoparticles is advantageous inasmuch as it offers unique properties due to their low particle size (such as their physical behavior when interacting or being irradiated with light), large surface area to volume ratio that may allow enhanced solubility compared to larger particles, tunable surface functionalization that facilitates the customization of ligands depending on the application, and specificity in the interaction with other entities such as viruses, prokaryotic cells and eukaryotic cells. Furthermore, nanoparticles can have intrinsic physical properties that can be advantageously applied as a therapy by themselves (i.e. plasmonic and magnetic nanoparticles for optical and magnetic hyperthermia, respectively).

Further, the resorbable nanoparticle is preferably an inorganic nanoparticle, i.e. a nanoparticle, comprising or consisting of an inorganic material. As mentioned in the following, the inorganic material is especially preferably a metal and/or a metal salt such as a metal oxide and/or metal hydroxide. In general, inorganic nanoparticles have unique chemical, electrical and optical effects and catalytic activities, which cannot be found in bulk metals. This facilitates a variety of fields of use, in particular with respect to therapy, diagnosis, drug delivery systems, biomedicine, photoelectrochemical devices, sensors, and the like.

In an embodiment of the invention, the resorbable nanoparticle comprises or includes at least one metal, in particular at least one elemental metal. Preferably, the at least one metal, in particular at least one elemental metal, is selected from the group consisting of magnesium, iron and zinc.

More preferably, the at least one metal, in particular at least one elemental metal, is magnesium or a magnesium alloy.

Magnesium is after sodium, potassium and calcium the most abundant cation in the human body. It is found both in bones and soft tissues playing key roles in enzymatic and cellular processes. Magnesium has the further advantage of a slow dissolution in a physiological aqueous environment releasing magnesium cations (Mg²⁺) that might form magnesium hydroxide (Mg(OH)₂). Chloride ions (Cl⁻) can further react with magnesium hydroxide to generate magnesium chloride (MgCl₂) which is highly soluble dissolving finally the magnesium. Thus, the ions released are totally biocompatible as they are Mg²⁺ ions, OH⁻ ions and Cl⁻ ions that can be effectively integrated or eliminated from the body as long as renal function is normal. Therefore, any toxicological risk can be avoided or at least reduced to a safe clinical level.

A further advantage of nanoparticles comprising or consisting of magnesium refer to their intrinsic optical properties such as their localized surface plasmon resonance (LSPR) which can be used for selective recognition events (i.e. immunorecognition, nucleic acid hybridization). Advantageously, nanoparticles comprising or consisting of magnesium show LSPR, in particular in the UV, visible and/or near-infrared region, in particular from 750 nm to 1200 nm, in particular 800 nm to 900 nm, preferably around 800 nm, that allows to use the transmission window of a biological tissue, in particular the tissue of a subject, without damaging it. This advantageously enables for the development of photothermal therapies and biomedical applications inside a human body or other mammalian body.

Furthermore, for example, magnesium oxide-based nanoparticles exhibit photocatalytic properties and therefore, they are able to generate reactive oxygen species mediated by light irradiation that can react with fluorescence probes or participate in polymerization reactions generating analytical signals that can be also related to selective recognition events. The presence of LSPR that can be excited at different wavelengths together with the generation of electron-hole pairs by using different excitation wavelengths opens the possibilities to develop multidetection platforms.

A further advantage of magnesium refers to luminescence which may be exploited in imaging, in particular medical imaging.

In the following table 1, methods for the synthesis of magnesium-based nanoparticles are listed:

TABLE 1
Methods for synthesizing magnesium-based nanoparticles

| Preparation | | Particle | |
|---|---|---|---|
| method | Precursors | size, nm | Reference |
| Hydrothermal | NH₃•H₂O, Mg(NO₃)₂ | 50-100 | Jiu et al. (2001) |
| Hydrothermal | Na₂CO₃, Mg(NO₃)₂ | 30 | Zhang (1999) |
| Hydrothermal | NH₃•H₂O, MgCl₂ | 62 | Zhu et al. (2001) |
| Hydrothermal | MgCl₂, NaOH | 15 | Suzuki et al. (1992) |
| Hydrothermal | Urea, MgCl₂ | 15-20 | Chen et al. (2002) |
| Microwave-assisted | Mg | 32-43 | Al-Gaashani (2012) |
| Microwave-assisted | MgSO₄, water/NaOH/CTAB/ethanol | 5 | Saoud (2014) |
| Sol-gel | Mg(OC₂H₆)₂•H₂O | 30 | Alvarado et al. (2000) |
| Sol-gel | Mg(NO₃)₂, stearic acid | 20-50 | Xu (2006) |
| Micro-emulsion | MgCl₂•6H₂O, Triton X-100 | 50-60 nm | Wu (2008) |
| Co-precipitaton | MgCl₂•6H₂O, NH4OH, PEG 12000 | 200 | Wang (2011) |
| Phytoassisted | Mg(NO₃)₂, S. chirayaita | 20 | Sharma (2017) |
| Fungal mediated | MgSO4.7H2O, A. niger | not shown | Hassan (2018) |

In the sol-gel method a magnesium alkoxide is hydrolyzed in an alcoholic media to produce the hydroxide with subsequent steps of hydrolysis, condensation, polymerization and thermal dehydration. Different parameters have to be optimized such as temperature, pH and the catalyst for gel formation (Alvarado, E., Tones-Martinez, L. M., Fuentes, A. F. and Quintana, P., Preparation and characterization of MgO powders obtained from different magnesium salt and the mineral dolomite. Polyhedron, 2000, 19, 2345-2351; X. M. Xu, Preparation and characterization of nanometer magnesia powder by electrochemical precipitation. Inorganic Chemicals Industry, 2006, 38, 32-34). This methodology is simple, cost effective, needs a low reaction temperature and is characterized by a high yield of nanoparticle production although it has the disadvantage of the high agglomeration rate of the MgO nanoparticles. Surfactant-mediated synthesis approaches have been studied to solve this problem such as the assisted method with cetyltrimethylammonium bromide (CTAB) which can also control the morphology and size of the nanoparticles. In the hydrothermal approach, a magnesium salt in basic aqueous media is mixed with a base solution at a varying ratio of Mg²⁺/OH⁻ being the obtained precipitate washed and calcined (J. P. Jiu, L. P. Li, Y. Ge, S. R. Zhang, F. Tu, A. R. Hua, and L. Nie, The preparation of MgO nanoparticles protected by polymer. Chinese Journal of Inorganic Chemistry, 2001, 17, 361-365; J. Zhang, Study on preparing nanometer-sized MgO by homogeneous precipitation. Method, Materials, 1999, 30, 193-194; Y. X. Zhu, R. J. Zeng, X. J. Liu, H. Zhang, R. Pan, X. L. Zhou, Y. Zhang, Preparation and characterization of MgO nanopowder, Journal of Xiamen University (Nature Science), 2001, 40, 1256-1258; M. Suzuki, M. Kagawa, Y. Syonoetal, Synthesis of ultrafine single-component oxide particles by the spray ICP technique, Journal of Material Science, 1992, 27, 679-684; G. R. Chen, S. H. Xu, J. Yang, The study of the preparing of nanometer MgO powder in the stearic acid gel method, Journal of Functional Materials, 2002, 35, 521-523). To control the morphology and size of the MgO nanoparticles the magnesium precursors, reactant solvents and calcination temperature have to be optimized. Starch and cellulose have been used as stabilizing agents. The microwave-assisted method, has the advantage of reducing reaction time, narrowing the size distribution and increasing the yield of the reaction (K. M. Saoud, S. Saeed, R. M. Al-Soubaihi and M. F. Bertino. American Journal Nanomaterials, 2014, 2, 21-25). In the micro-emulsion method, the precursor is heated with the surfactant to get the oxide and the size and morphology of the nanoparticles are controlled by the choice of the surfactant, the concentration of water, non-polar phase and surfactant (J. Wu, H. Yana, X. Zhang, L. Wei, X. Liu, Bingshe Xu. Magnesium hydroxide nanoparticles synthesized in water-in-oil microemulsions. Journal of Colloid and Interface Science, 2008, 324, 167-171). The co-precipitation methods are also an alternative to prepare magnesium nanoparticles under mild conditions (E. R. H. Walter, M. A. Fox, D. Parker, J. A. G. Williams. Enhanced selectivity for Mg2+ with a phosphinate-based chelate: APDAP versus APTRA. Dalton Transactions, 2018, 47, 1879-1887).

Further, the resorbable nanoparticle may comprise at least one compound including magnesium, in particular ionic magnesium. In particular, the resorbable nanoparticle may consist of such a compound. Preferably, the least one compound including magnesium, in particular ionic magnesium, is a metal salt, in particular as detailed in the following embodiment.

In a further embodiment of the invention, the resorbable nanoparticle comprises at least one metal salt, in particular at least one metal oxide and/or at least one metal hydroxide. Preferably, the at least one metal oxide is selected from the group consisting of magnesium oxide, iron oxide, zinc oxide and mixtures of at least two of the afore-mentioned metal oxides, and/or the at least one metal hydroxide is preferably selected from the group consisting of magnesium hydroxide, iron hydroxide, zinc hydroxide and mixtures of at least two of the afore-mentioned metal hydroxides. In particular, the resorbable nanoparticle may consist of at least one metal salt, in particular at least one metal oxide and/or at least one metal hydroxide, wherein the at least one metal oxide is preferably selected from the group consisting of magnesium oxide, iron oxide, zinc oxide and mixtures of at least two of the afore-mentioned metal oxides, and/or the at least one metal hydroxide is preferably selected from the group consisting of magnesium hydroxide, iron hydroxide, zinc hydroxide and mixtures of at least two of the afore-mentioned metal hydroxides.

More preferably, the at least one metal oxide is magnesium oxide and/or the at least one metal hydroxide is magnesium hydroxide.

In a further embodiment of the invention, the resorbable nanoparticle comprises a core-sheath structure, wherein the core comprises the at least one metal, in particular at least one elemental metal, and/or the sheath comprises the least one metal salt, in particular at least one metal oxide and/or at least one metal hydroxide. In particular, the resorbable nanoparticle may comprise a core-sheath structure, wherein the core consists of the at least one metal, in particular at least one elemental metal, and/or the sheath consists of the least one metal salt, in particular at least one metal oxide and/or at least one metal hydroxide. The core-sheath structure may have a diameter, in particular average diameter, from 0.1 nm to 500 nm, in particular 1 nm to 100 nm, preferably 10 nm to 50 nm. Further, the sheath of the core-sheath structure may have a thickness from 0.05 nm to 250 nm, in particular 0.5 nm to 50 nm, preferably 5 nm to 25 nm. Especially preferably, the metal, in particular elemental metal, is magnesium, and the metal salt is magnesium oxide and/or magnesium hydroxide. More preferably, the core may comprise or consist of elemental magnesium and the sheath may comprise or consist of magnesium oxide and/or magnesium hydroxide.

In a further embodiment of the invention, the resorbable nanoparticle has an average diameter, in particular determined by means of Transmission Electron Microscopy (TEM), from 0.1 nm to 500 nm, in particular 1 nm to 100 nm, preferably 10 nm to 50 nm, in particular including any integer and/or decimal number included in the afore-mentioned ranges.

Further, the resorbable nanoparticle may have a specific surface area (SSA), in particular determined by means of Brunauer-Emmett-Teller (N2-BET) adsorption method, methylene blue (MB) staining, ethylene glycol monoethyl ether (EGME) adsorption, electrokinetic analysis of complex-ion adsorption or Protein Retention (PR) method or according to ISO standard 9277, from 5 m²/g to 800 m²/g, in particular 25 m²/g to 400 m²/g, preferably 50 m²/g to 80 m²/g.

In principle, the resorbable nanoparticle may have a polyhedral shape or a non-polyhedral shape.

In a further embodiment of the invention, the resorbable nanoparticle has a shape selected from the group consisting of a cube shape, a cuboid shape, a prism shape, a tetrahedral shape, a cylindrical shape, a triangular shape, a pyramidal shape, a cone shape, an egg shape, a spherical shape, a star shape, a rod shape and combinations of at least two of the afore-mentioned shapes.

In a further embodiment of the invention, the at least one compound or a moiety thereof is attached, in particular covalently attached (conjugated) and/or non-covalently attached, in particular by means of van der Waals forces and/or hydrogen bonds and/or coordinative bonds and/or ionic interactions, to the resorbable nanoparticle or a surface of the resorbable nanoparticle, preferably forming a coating or layer, more preferably an outer coating or outer layer, of or onto the resorbable nanoparticle or onto the surface of the resorbable nanoparticle. The coating or layer may have a thickness from 0.1 nm to 100 nm, in particular 1 nm to 100 nm, preferably 5 nm to 50 nm.

In a further embodiment of the invention, the least one compound or a moiety thereof is attached directly or indirectly to the surface of the resorbable nanoparticle.

In a further embodiment of the invention, the at least one compound is selected from the group consisting of an antioxidant agent, a capping agent, an antibody such as a monoclonal antibody, a protein, a nucleic acid, a lipid, an antigen, an agent being capable of binding to a pathogen and combinations of at least two of the afore-mentioned compounds.

The term “antioxidant agent” as used according to the present invention refers to a compound which is capable of preventing the resorbable nanoparticle from being oxidized.

Preferably, the antioxidant agent is an unsaturated fatty acid, in particular selected from the group consisting of oleic acid, elaidic acid, palmitoleic acid, linoleic acid, linolelaidic acid, gamma-linolenic acid, alpha-linolenic acid and mixtures of at least two of the afore-mentioned unsaturated fatty acids. Oleic acid is especially preferred.

The term “capping agent” as used according to the present invention refers to a compound which is able capable of preventing the resorbable nanoparticle from growing, in particular by means of agglomeration, or retarding a growing of the resorbable nanoparticle, in particular by means of agglomeration. According to the present invention, the capping agent may also be termed as “capping ligand”.

Preferably, the capping agent is selected from the group consisting of catecholates, salicylic acid, salicylates, phosphates, phosphonates, bisphosphonates, hydrophilic polymers, in particular conjugated hydrophilic polymer and mixtures of at least two of the afore-mentioned capping agents. More preferably, the capping agent may be selected from the group consisting of dopamine, 3,4-dihydroxyhydrocinnamic acid, 2-aminoethylphosphonic acid, alendronate, alendronic acid, Furaptra (Mag-Fura-2) and combinations of at least two of the afore-mentioned capping agents. Preferably, the alendronate is sodium alendronate, in particular in a hydrate form, preferably trihydrate form.

The term “catecholates” as used according to the present invention refers to compounds carrying or bearing at least one catechol moiety or skeleton, i.e. at least one 1,2-dihydroxybenzene moiety or skeleton and/or at least one 1-hydroxy,2-alkoxybenzene moiety or skeleton and/or at least one 1-alkoxy,2-hydroxybenzene moiety or skeleton and/or at least one 1,2-dialkoxybenzene moiety or skeleton.

The term “salicylates” as used according to the present invention refers to salts of salicylic acid and/or compounds in particular carrying or bearing at least one salicylic acid moiety or skeleton and/or at least one salicylic acid ester moiety or skeleton and/or at least one salicylic acid amide moiety or skeleton.

The term “hydrophilic polymers” as used according to the present invention refers to a polymer being soluble or swellable in water or an aqueous solution.

Advantageously, by using a hydrophilic polymer, in particular as detailed in the following, the stability of the resorbable nanoparticle, and thus of the inventive nanoparticle material, in particular when being dispersed, preferably in an aqueous media, may be increased.

In particular, the hydrophilic polymer may be a linear and/or multifunctional, in particular bifunctional, preferably a linear and multifunctional, in particular bifunctional, polymer. Advantageously, the hydrophilic polymer may have two terminal functional groups, namely a first terminal functional group and a second terminal functional group. The first terminal functional group is capable of binding, in particular covalently and/or non-covalently binding, to the resorbable nanoparticle or the surface thereof. The first functional group may also be termed as “anchoring group” within the scope of the present invention. Preferably, the second terminal functional group is capable of binding, in particular covalently and/or non-covalently binding, (directly) to a pathogen or to an agent being capable of binding to a pathogen or to an analyte. Further, the first terminal functional group may be a functional group selected from the group consisting of carboxyl group, hydroxyl group, amine group, phosphate group, phosphonate group, sulphonate group and mixtures of at least two of the afore-mentioned functional groups. Further, the second terminal functional group may be a chelating group, in particular a multidentate ligand group such as a bidentate, tridentate, tetradentate or octadentate ligand group. For example, the chelating group may comprise at least one functional group selected from the group consisting of carboxyl group, hydroxyl group, amine group, phosphate group, phosphonate group, sulfonate group and mixtures of at least two of the afore-mentioned functional groups. For example, the chelating group may be a phosphinate-based chelating group such as o-aminophenol-N,N,O-triacetate (APTRA) moiety or an o-aminophenol-N,N-diacetate-O-methylene-methylphosphinate (APDAP) moiety.

Specifically, the hydrophilic polymer may comprise a polymer block or unit that is selected from the group consisting of polyethylene glycol block or unit, poly-aspartic acid block or unit, poly-glutamic acid block or unit, a hydrophilic protein block or unit such an albumin block or unit, in particular serum album block or unit, for example bovine serum albumin (BSA) block or unit, and mixtures of at least two of the afore-mentioned polymer bocks or units.

Preferably, the hydrophilic polymer may be a polymer selected from the group consisting of polyethylene glycol conjugated with a catecholate, polyethylene glycol conjugated with a salicylate, polyethylene glycol conjugated with a phosphate, polyethylene glycol conjugated with a phosphonate, polyethylene glycol conjugated with a bisphosphonate, poly-glutamic acid conjugated with a catecholate, poly-glutamic acid conjugated with a salicylate, poly-glutamic acid conjugated with a phosphate, poly-glutamic acid conjugated with a phosphonate, poly-glutamic acid conjugated with a bisphosphonate, poly-aspartic acid conjugated with a catecholate, poly-aspartic acid conjugated with a salicylate, poly-aspartic acid conjugated with a phosphate, poly-aspartic acid conjugated with a phosphonate, poly-aspartic acid conjugated with a bisphosphonate and mixtures of at least two of the afore-mentioned hydrophilic polymers.

More preferably, the hydrophilic polymer may be a polymer selected from the group consisting of polyethylene glycol conjugated with dopamine, polyethylene glycol conjugated with 3,4-dihydroxyhydrocinnamic acid, polyethylene glycol conjugated with 2-aminoethylphosphonic acid, polyethylene glycol conjugated with alendronate, polyethylene glycol conjugated with Furaptra (Mag-Fura-2), poly-glutamic acid conjugated with dopamine, poly-glutamic acid conjugated with 3,4-dihydroxyhydrocinnamic acid, poly-glutamic acid conjugated with 2-aminoethylphosphonic acid, poly-glutamic acid conjugated with alendronate, poly-glutamic acid conjugated with Furaptra (Mag-Fura-2), poly-aspartic acid conjugated with dopamine, poly-aspartic acid conjugated with 3,4-dihydroxyhydrocinnamic acid, poly-aspartic acid conjugated with 2-aminoethylphosphonic acid, poly-aspartic acid conjugated with alendronate, poly-aspartic acid conjugated with Furaptra (Mag-Fura-2) and mixtures of at least two of the afore-mentioned hydrophilic polymers.

The term “polyethylene glycol” as used according to the present invention may refer to an unfunctionalized polyethylene glycol, i.e. a polyethylene glycol carrying or bearing two terminal hydroxy groups, or to a functionalized polyethylene glycol, i.e. to a polyethylene glycol having at least one terminal hydroxy group, in particular only one terminal hydroxy group or both terminal hydroxy groups to be replaced by a different functional group such as carboxyl group or a carboxylate group. Preferably, the term “polyethylene glycol” as used according to the present invention means a carboxylated polyethylene glycol, i.e. a polyethylene glycol carrying or bearing at least one terminal carboxyl or carboxylate group, in particular only one terminal carboxyl or carboxylate group or two terminal carboxyl or carboxylate groups. Further, the polyethylene glycol according to the present invention may have a molecular weight of 500 Da to 50000 Da, in particular 1000 Da to 10000 Da, preferably 1500 Da to 5000 Da, for example 3000 Da.

The term “agent being capable of binding to a pathogen” may also be termed as “targeting ligand to a pathogen” within the scope of the present invention, thereby emphasizing that such an agent and ligand, respectively forms a binding target for a pathogen resulting in formation of a binding between the targeting ligand and the pathogen.

The antibody (mentioned in the context of the at least one compound) may be selected from the group consisting of immunoglobulin G1 (IgG1), immunoglobulin 3 (IgG3), immunoglobulin M (IgM), immunoglobulin A (IgA), monoclonal antibodies (mAbs) targeting the receptor-binding motif (RBM) of ACE2, monoclonal antibodies (mAbs) targeting CR3022 cryptic site (which is the most frequent epitope targeted by cross-neutralizing antibodies (i.e. COVA1-16, H014, EY6A and ADI-56046)), monoclonal antibodies (mAbs) targeting 5309 binding site and mixtures of at least two of the afore-mentioned antibodies. Preferably, the antibody is directed against a pathogen such as SARS-CoV-2 or a part, in particular protein, thereof such as spike protein, in particular spike S protein.

The protein (mentioned in the context of the at least one compound) may be a receptor-binding domain (RBD), in particular of a pathogen such as SARS-CoV-2. For example, the protein may be SARS-CoV-2 S-receptor-binding-domain. Alternatively, the protein may be ACE 2 receptor protein.

Further, the agent being capable of binding to a pathogen (mentioned in the context of the at least one compound) may be selected from the group consisting of an antibody, a protein, a nucleic acid, a lipid, an antigen and combinations of at least two of the afore-mentioned agents being capable of binding to a pathogen. With respect to more details concerning the afore-mentioned agents being capable of binding to a pathogen, reference is made in its entirety to the previous description. In particular, the antibodies and proteins previously mentioned may also be agents being capable of binding to a pathogen according to the present invention.

In a further embodiment of the invention, the at least one compound comprises or means a capping agent and an agent being capable of binding to a pathogen. With respect to further details concerning the capping agent and the agent being capable of binding to a pathogen, reference is made in its entirety to the previous description. The capping agents and agents being capable of binding to a pathogen previously mentioned may also be capping agents and agents being capable of binding to a pathogen according to this embodiment.

In a further embodiment of the invention, the capping agent or a moiety thereof is directly attached to the resorbable nanoparticle or the surface of the resorbable nanoparticle, and the agent being capable of binding to a pathogen or a moiety of the agent being capable of binding to a pathogen is attached to the capping agent or a moiety thereof. In other words, the agent being capable of binding to a pathogen or a moiety of the agent being capable of binding to a pathogen is preferably indirectly, i.e. via the capping agent or a moiety thereof, attached to the resorbable nanoparticle or to the surface of the resorbable nanoparticle. Specifically, the capping agent or a moiety thereof may be covalently and/or non-covalently, in particular by means of van der Waals forces and/or hydrogen bonds and/or coordinative bonds and/or ionic interactions, attached to the resorbable nanoparticle or to the surface of the resorbable nanoparticle. Further, the agent being capable of binding to a pathogen or a moiety of the agent being capable of binding to a pathogen may be covalently and/or non-covalently, in particular by means of van der Waals forces and/or hydrogen bonds and/or coordinative bonds and/or ionic interactions, attached to the capping agent or a moiety thereof.

In a further embodiment of the invention, the nanoparticle material is for use in preventing and/or treating, i.e. prevention and/or treatment of, a disease and/or a disorder in a subject or for use in a method of prevention and/or treatment of a disease and/or a disorder in a subject, wherein the method comprises the step of administering the nanoparticle material. Preferably, the disease and/or disorder is caused by a pathogen. The pathogen may preferably be of fungal, viral or bacterial origin, i.e. may have a fungal, viral or bacterial origin. Preferably, the pathogen is of viral origin.

The term “subject” as used according to the present invention may mean a human being or a non-human mammal, for example a horse, a cow, a dog, a cat, a rabbit, a rat or a mouse. Preferably, the term “subject” as used according to the present invention means a human being or human patient.

Preferably, the disease and/or disorder caused by a pathogen may be an infectious disease, in particular a fungal, viral or bacterial infectious disease.

For example, the disease or disorder caused by a pathogen may be selected from the group consisting of coronavirus disease 2019 (COVID-19), chickenpox, common cold, diphtheria, E. coli, giardiasis, HIV/AIDS, infectious mononucleosis, influenza (flu), Lyme disease, malaria, measles, meningitis, mumps, poliomyelitis (Polio), pneumonia, Rocky Mountain Spotted Fever, rubella (German measles), salmonella infections, Severe Acute Respiratory Syndrome (SARS), sexually transmitted diseases, shingles (Herpes zoster), tetanus, toxic shock syndrome, tuberculosis, viral hepatitis, West Nile virus and whooping cough (pertussis).

Preferably, the disease and/or disorder caused by a pathogen is coronavirus disease 2019 (COVID-19), i.e. a contagious disease caused by severe acute respiratory syndrome coronavirus 2 (SARS-CoV-2).

In a further embodiment of the invention, the pathogen of viral origin is SARS-CoV-2 or a virus of coronavirus type such as severe acute respiratory syndrome coronavirus [1] (SARS-CoV[−1]) or Middle East respiratory syndrome coronavirus (MERS-CoV).

Further, the nanoparticle material may be administered topically, dermally, orally or parenterally, in particular intravenously, intradermally, intramuscularly, intraperitoneally, intra-arterially, nasally or transmucosally.

Further, in particular for preventing and/or treating the disease and/or disorder, preferably caused by the pathogen, the nanoparticle material may be irradiated with light, in particular UV light (ultraviolet light), in particular having a wavelength from 100 nm to 380 nm, and/or visible light (VIS light), in particular having a wavelength from >380 nm to 750 nm, preferably 400 nm to 750 nm, and/or UV-Vis light (ultraviolet-visible light), in particular having a wavelength from 100 nm to 750 nm, and/or near infrared light (NIR light), in particular having a wavelength from 750 nm to 1200 nm, preferably >750 nm to 1200 nm, in particular 800 nm to 900 nm. More preferably, the nanoparticle material may be irradiated with light having a wavelength from 100 nm to 1200 nm, in particular 100 nm to 800 nm or 750 nm to 1200 nm, preferably 400 nm to 800 nm or 800 nm to 900 nm. Thus, the temperature of the nanoparticle material may be advantageously increased, which in turn may result in inactivation or destruction of the pathogen.

In a further embodiment of the invention, the nanoparticle material is for use in preventing and/or treating, i.e. prevention and/or treatment of, a disease and/or a disorder in a subject or for use in a method of prevention and/or treatment of a disease and/or a disorder in a subject, wherein the method comprises the step of administering the nanoparticle material, wherein the disease and/or disorder is a proliferative disease.

In a further embodiment of the invention, the proliferative disease is a disease associated with, in particular at least some degree, of abnormal cell proliferation. The proliferative disease may be a benign proliferative disease or a malignant proliferative disease. Preferably, the proliferative disease is a cancer, in particular selected from the group consisting of carcinoma, lymphoma, blastoma, sarcoma, leukemia, non-localized cancer, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, skin cancer, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulvar cancer, thyroid cancer, hepatic carcinoma, head cancer and neck cancer.

Further, the nanoparticle material may be administered topically, dermally, orally or parenterally, in particular intravenously, intradermally, intramuscularly, intraperitoneally, intra-arterially, nasally or transmucosally.

Further, in particular for preventing and/or treating the proliferative disease, the nanoparticle material may be irradiated with light, in particular UV light (ultraviolet light), in particular having a wavelength from 100 nm to 380 nm, and/or visible light (VIS light), in particular having a wavelength from >380 nm to 750 nm, preferably 400 nm to 750 nm, and/or UV-Vis light (ultraviolet-visible light), in particular having a wavelength from 100 nm to 750 nm, and/or near infrared light (NIR light), in particular having a wavelength from 750 nm to 1200 nm, preferably >750 nm to 1200 nm, in particular 800 nm to 900 nm. More preferably, the nanoparticle material may be irradiated with light having a wavelength from 100 nm to 1200 nm, in particular 100 nm to 800 nm or 750 nm to 1200 nm, preferably 400 nm to 800 nm or 800 nm to 900 nm. Thus, the temperature of the nanoparticle material may be advantageously increased, which in turn may result in inactivation or destruction of a proliferative cell, in particular tumoral cell.

Further, the nanoparticle material according to the present invention may be for use in diagnosing, i.e. diagnosis of, a disease and/or a disorder in a subject. Preferably the disease and/or disorder is caused by a pathogen, in particular of fungal, viral or bacterial origin, or is a proliferative disease. With respect to further details of the disease and/or disorder and the pathogen, reference is made in its entirety to the previous description.

In a further embodiment of the invention, the nanoparticle material is for use in imaging applications, in particular non-medical imaging applications or medical imaging applications. Preferably, the nanoparticle material is for use in medical imaging.

The term “medical imaging” as used according to the present invention refers to a technique and process of imaging the interior of a body for clinical analysis and/or medical intervention and/or for visual representation of the function of organs or tissues.

The medical imaging may be selected from the group consisting of radiology, X-ray radiography, magnetic resonance imaging, ultrasound, endoscopy, elastography, tactile imaging, thermography, medical photography, nuclear medicine functional imaging, positron emission tomography (PET) and single-photon emission computed tomography (SPECT).

In a further embodiment of the invention, the nanoparticle material is for use in generating radicals, in particular by light irradiation or mediated by light irradiation, preferably for treating a disease and/or a disorder in a subject. More preferably, the disease and/or disorder is a disease and/or disorder caused by a pathogen, in particular of fungal, viral or bacterial origin, or a proliferative disease. With respect to further details of the disease and/or disorder and the pathogen, reference is made in its entirety to the previous description.

In a further embodiment of the invention, the nanoparticle material is for use in disinfecting, i.e. disinfection of, surfaces, in particular of medical devices such as surgical instruments and/or surgical implants, in particular selected from the group consisting of surgical sutures, arterial prostheses, venous prostheses, stents, stent grafts, wound dressings, surgical meshes, wound fixing devices, catheters such as balloon catheters, hip prostheses and knee prostheses.

In a further embodiment of the invention, the nanoparticle material is for use in detecting, i.e. detection of, a pathogen or for developing a procedure for detecting, i.e. detection of, a pathogen. Preferably, the pathogen is of fungal, viral or bacterial origin. Preferably, the pathogen is a coronavirus, in particular selected from the group consisting of SARS-CoV-2, SARS-CoV[−1] and MERS-CoV.

In a further embodiment of the invention, the nanoparticle material is for use in detecting, i.e. detection of, an analyte or for developing a procedure for detecting, i.e. detection of, an analyte. The analyte may be selected from the group consisting of an antibody, a protein, a tumor marker, a nucleic acid, a small molecule or a combination of at least two of the afore-mentioned analytes. The antibody may be an antibody against a pathogen. The protein may be a protein associated to a pathogen such as a spike protein, in particular of SARS-CoV-2.

In a further embodiment of the invention, the nanoparticle material is for use in developing a sensor or sensor technology.

A second aspect of the present invention refers to a process for obtaining a nanoparticle material according to the first aspect of the invention. The process comprises the steps of:
a) coating a resorbable nanoparticle with an antioxidant agent and
b) replacing the coating of the antioxidant agent by functionalizing the resorbable nanoparticle with at least one compound being different from the antioxidant agent.

Preferably, step a) is carried out in the presence of an organic solvent. More specifically, step a) may be carried out by forming a dispersion containing the resorbable nanoparticle, antioxidant agent and organic solvent. The organic solvent may be in particular selected from the group consisting of alcohols such as methanol and/or ethanol and/or isopropanol, saturated hydrocarbures such as hexane, insaturated, hydrocubures and mixtures of at least two of the afore-mentioned organic solvents. More specifically, the dispersion containing the resorbable nanoparticle, antioxidant agent and organic solvent may be formed by means of sonification.

Further, step a) may be carried out by using an unsaturated fatty acid, in particular oleic acid, as an antioxidant agent. With respect to further useful unsaturated fatty acids, reference is made in its entirety to the previous description. The unsaturated fatty acids mentioned there may also be used for the process according to the second aspect of the present invention.

By carrying out step a), the resorbable nanoparticle may be advantageously prevented from being oxidized.

Preferably, step b) is carried out by using an aqueous liquid, in particular aqueous solution, containing the least one compound being different from the antioxidant agent. More specifically, the above-mentioned dispersion containing the resorbable nanoparticle, antioxidant agent and organic solvent and the aqueous liquid, in particular aqueous solution, containing the least one compound being different from the antioxidant agent are incubated, in particular at room temperature, i.e. 15° C. to 30° C., preferably 20° C. to 25° C., and/or for 0.1 min to 3600 min, preferably 1 min to 60 min.

Further, step b) may be carried out by using a capping agent. With respect to useful capping agents, reference is made in its entirety to the previous description. The capping agents mentioned there may also be used for the process according to the second aspect of the present invention.

By carrying out step b), the resorbable nanoparticle may be advantageously prevented from growing, in particular by means of agglomeration.

The process may further comprise a step c) isolating and/or purifying the resorbable nanoparticle being functionalized with the least one compound being different from the antioxidant agent from an aqueous phase that has formed during step b).

With respect to further features and advantages, in particular in terms of the resorbable nanoparticle and/or antioxidant agent and/or least one compound being different from the antioxidant agent, reference is made in its entirety to the respective embodiments made under the first aspect of the present invention. The respective features and advantages disclosed there do apply, mutatis mutandis, with respect to the process according to the second aspect of the present invention.

According to a third aspect, the present invention refers to a product comprising and/or being coated with a resorbable functionalized nanoparticle according to the first aspect of the invention.

The product may be selected from the group consisting of a pharmaceutical composition, a medical device, a medical kit, a drug delivery system, photoelectrochemical device and a sensor.

The pharmaceutical composition may further comprise a pharmaceutically acceptable vehicle, diluent, excipient or carrier. The pharmaceutically acceptable vehicle, diluent, excipient or carrier can be any compound or combination of compounds which enables administration of the nanoparticle material within the pharmaceutical composition. Preferably, the pharmaceutically acceptable vehicle, diluent, excipient or carrier is in the form of an emulsion, an aqueous solution, a buffer, a lipid or any other suitable compound or composition, in particular suitable for perfusion or instillation.

The medical device may be in particular a surgical instrument or surgical implant, in particular selected from the group consisting of a surgical suture, an arterial prosthesis, a venous prosthesis, a stent, a stent graft, a wound dressing, a surgical mesh, a wound fixing device, a catheter such as a balloon catheter, a hip prosthesis and a knee prosthesis.

With respect to further features and advantages of the product, in particular with respect to the nanoparticle material, reference is made in its entirety to the respective features and advantages disclosed under the first aspect of the invention. These features and advantages do apply, mutatis mutandis, with respect to the product according to the third aspect of the invention.

Further features and advantages of the invention will become clear from the following examples. The individual features can be realized either singularly or severally in combination in one embodiment of the invention. The preferred embodiments only serve for illustration and better understanding of the invention and are not to be understood as in any way limiting the invention.

### BRIEF DESCRIPTION OF THE FIGURES

For a better understanding of what has been disclosed, some figures are attached which schematically or graphically and solely by way of non-limiting example show a practical case of embodiments of the present invention.

FIG. 1 graphically shows a Raman spectrum of magnesium oxide nanopowder provided by Sigma.

FIG. 2 graphically shows UV-Vis spectra of dispersed magnesium oxide nanopowder.

FIG. 3 graphically shows generation of reactive oxygen species and hydroxyl radical from magnesium oxide nanopowder after UV irradiation.

FIG. 4 graphically shows yield of magnesium oxide nanomaterial dissolution after overnight incubation in different conditions. The magnesium oxide concentration was 1 mg mL⁻¹.

FIG. 5 shows capping agents for functionalization of magnesium oxide nanoparticles.

FIG. 6 shows coupling reaction schemes of capping ligands to PEG.

FIG. 7 graphically shows the dispersion yield of magnesium oxide nanomaterial with different ligands.

FIG. 8 graphically shows labeling of magnesium oxide nanomaterials with fluorescence probes.

### DETAILED DESCRIPTION

### Experiments

### 1. Raw Material for Magnesium Oxide Nanoparticles

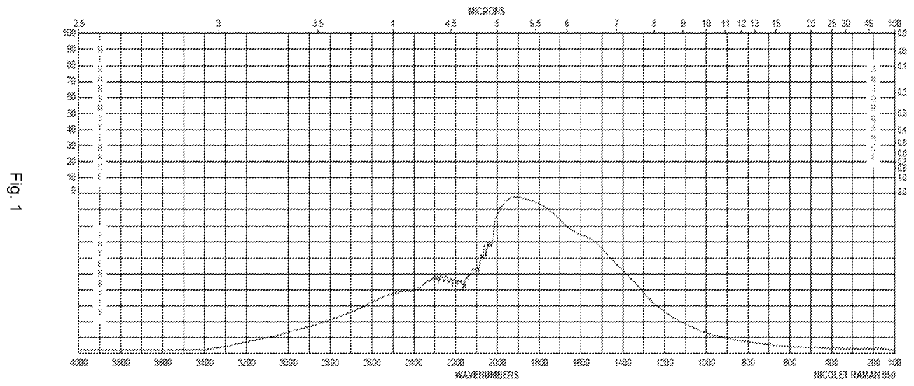

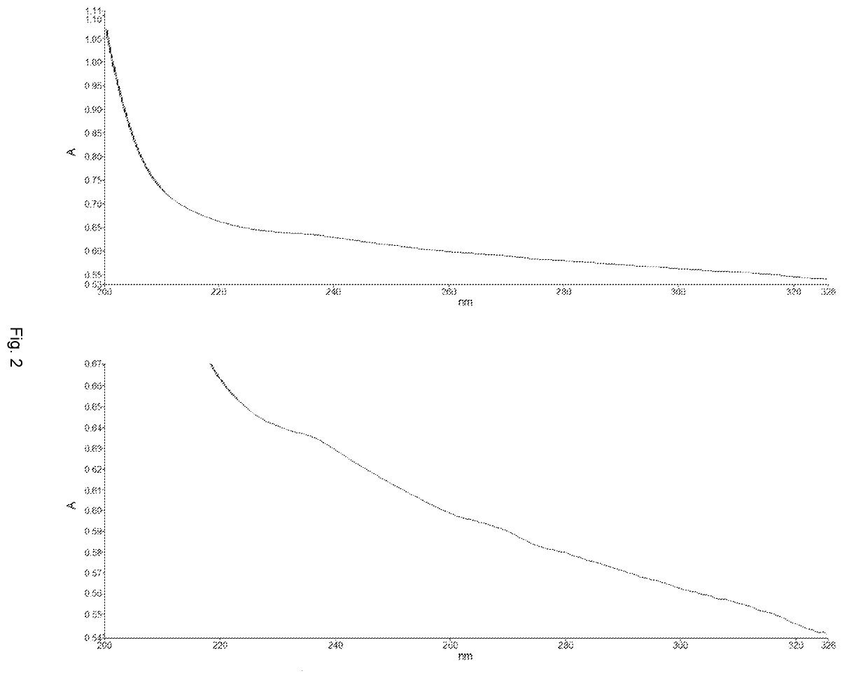

Magnesium oxide nanoparticles were purchased from Sigma (catalogue number 549649-5G, nanopowder, ≤50 nm particle size). Sigma provided the value of the specific surface area (50-80 m² g⁻¹) and the Raman spectra of the product (see FIG. 1).

### 2. Characterization of Functionalized Nanoparticles

2.1. UV-Vis-NIR Characterization

The UV-Vis-NIR spectra of the raw material (magnesium oxide nanoparticles) was obtained. The raw material was dispersed in aqueous media after sonication in water bath for 30 min of a suspension of magnesium oxide nanopowder at 20 mg mL⁻¹ in water. The suspension was not completely stable and tended to sediment in few hours although some dispersed material remained in suspension. The UV-Vis-NIR spectra of the dispersed nanoparticles was obtained in order to determine the position of the surface plasmon resonance.

The UV-Vis spectrum showed a shoulder at around 235 nm that could correspond to the surface plasmon of the magnesium oxide nanoparticles. Ideally, magnesium-based nanoparticles should exhibit a localized surface plasmon resonance (LSRP) in the near infrared region, particularly at around 800 nm, for the biomedical purposes as this is the biological window where tissues (i.e. biological tissues) exhibit maximum transparency.

2.2. Generation of Reactive Oxygen Species (ROS) Induced by Light

The generation of reactive oxygen species was evaluated by fluorescent probes. DFC-DA is a non-selective fluorescent probe for reactive oxygen species. Hydroxyphenylfluorescein (HPF) selectively detects highly reactive oxygen species (hROS) such as hydroxyl radical (·OH) and peroxynitrite (ONOO—), whereas it does not react with other reactive oxygen species (for example, superoxide and hydrogen peroxide).

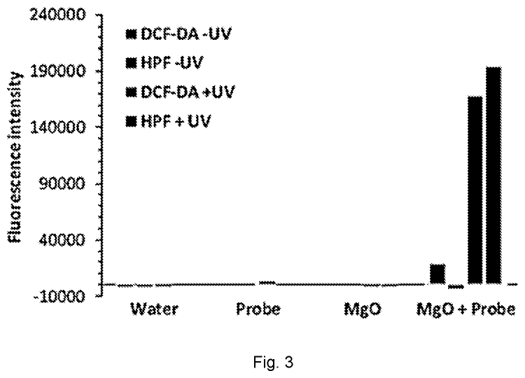

Magnesium oxide nanopowder was pressed in 24-well plates and incubated with the selected fluorescence probes. The generation of ROS species was evaluated after irradiation with UV light. As it can be seen in FIG. 3, ROS species and hydroxyl radicals were generated after UV exposure. Controls of magnesium oxide nanopowder without UV irradiation were also prepared together with the control probes without interaction with the magnesium oxide material.

2.3. Bacteria/Cell Studies

Cell viability of treated cells were evaluated by different assays (Tripan blue, MTT). Antibacterial efficiency were also evaluated by the corresponding assays. Cells and bacteria were incubated with magnesium oxide nanoparticles and then irradiated with light according to their surface plasmon resonance position. Controls was also prepared with cells and bacteria without previous incubation with magnesium oxide nanoparticles and with incubation with magnesium oxide nanoparticles but without light irradiation.

2.4. Dissolution of Magnesium Oxide Nanoparticles in Biological Environments

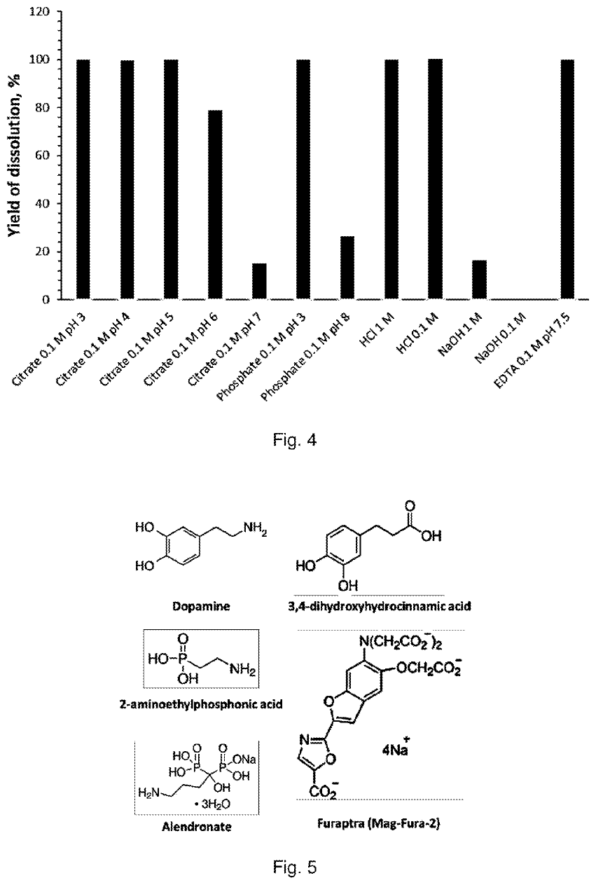

Magnesium oxide nanoparticles were incubated in physiological conditions to assess their dissolution in these environments. Incubation in phosphate saline buffer and at endosome/lysosome pHs was carried out to consider a possible degradation route in biological conditions. After the incubation in these conditions, the released magnesium was quantified by ICP-MS. As it can be seen in FIG. 4, magnesium oxide nanomaterial was dissolved at pH below 5 which is pH found in endosomes and lysosomes probing that magnesium oxide nanoparticles can be degraded after cell internalization if the mechanisms of entry and intracellular trafficking render these nanomaterials inside these organelles.

### 3. Procedures for the Functionalization of Magnesium Oxide Nanoparticles

The following procedures were used for the functionalization of magnesium oxide nanoparticles:
Coating with capping ligands with affinity for magnesium oxide rendering hydrophilic nanoparticles
Pre-dispersion with oleic acid and transfer to aqueous phase
Pre-dispersion with oleic acid and oxidation to transfer to aqueous phase
Functionalization with capping ligands with affinity for magnesium oxide and derivatization with PEG/proteins to increase the stability of the dispersed nanoparticles

3.1. Functionalization with Capping Ligands with Affinity for Magnesium Oxide

Different capping ligands were selected for the functionalization of magnesium oxide nanoparticles (see FIG. 5). This method is applicable to ligands whose anchoring groups forms stable magnesium complexes such as catecholates, salicylates, phosphates and phosphonates. Even if the affinity of each individual group for the particle surface is low, the multiplicity of anchoring groups provides a strong adsorption. For example, poly-aspartic and poly-glutamic acids were tested for the functionalization of magnesium oxide nanoparticles. In this procedure, the capping ligands were sonicated with the nanoparticles until a complete dispersion was obtained. The stability of the dispersion was evaluated by the absorption spectra of the obtained mixture.

As a starting point for the optimization of the procedure, the magnesium oxide nanoparticles (200 mg) were dispersed in 25 mL of 1% capping agent with continuous stirring at different times. The concentration of nanoparticles and capping agent in the dispersion mixture together with the incubation temperature was optimized to increase the stability of the dispersed nanoparticles. The excess of capping agent was removed by centrifugation and washing with water.

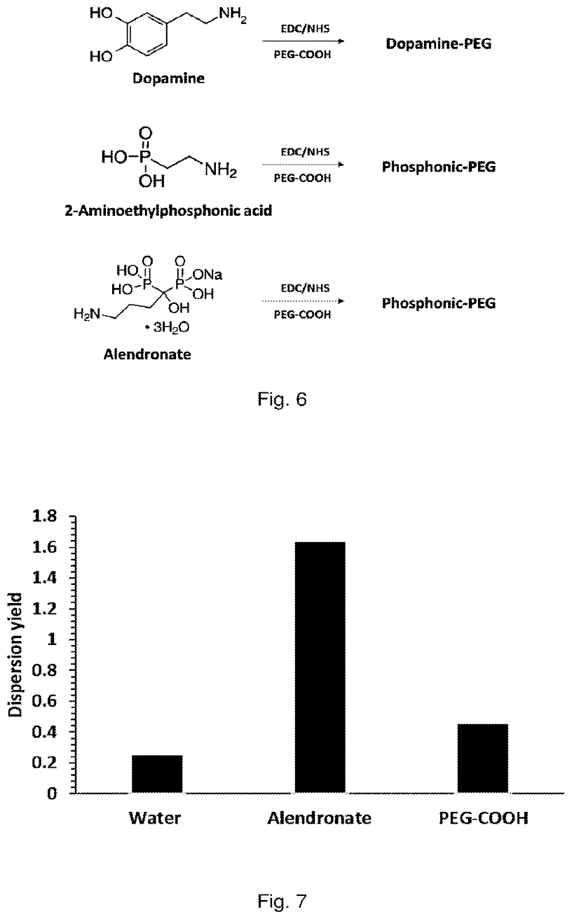

The possibility to conjugate these capping ligands (catecholates, phosphonates, bisphosphonates) to hydrophilic polymers was also evaluated. The resulting conjugate was also used for coating the magnesium oxide nanoparticles with the advantage of providing a polymeric hydrophilic coating that could increase the stability of the dispersed nanoparticles. The selected hydrophilic polymers were polyethylene glycol (PEG, Hydroxyl-PEG-COOH Sigma 670812, molecular weight 3000 Da), poly-glutamic acid and poly-aspartic acid. BSA could be also used for this approach. The carboxyl groups of these polymers were conjugated to dopamine, 2-aminoethylphosphonic acid or alendronate by carbodiimide coupling reaction (see FIG. 6 for PEG derivatization as an example). The activated carboxyl group reacted with the amine group of the capping ligands. The excess of reagents was removed by molecular exclusion chromatography. The conjugated PEG was sonicated with magnesium oxide nanoparticles to disperse the material. The stability of the dispersion was evaluated by the absorption spectra of the obtained mixture.

3.2. Pre-Dispersion of Nanoparticles with Oleic Acid and Transference to Aqueous Phase

Metal oxide nanoparticles are usually dispersed with oleic acid in non-aqueous media. In this approach magnesium oxide nanoparticles were first coated with oleic acid in order to disperse them in non-aqueous media and then, the coating was replaced by a hydrophilic coating to transfer the nanoparticles to aqueous media. The surface functionalization by this biphasic protocol avoids the oxidation and agglomeration of the nanoparticles. In this approach, nanoparticles were first dispersed in oleic acid in an organic phase such as methanol or hexane. As a starting procedure, 100 mg of magnesium oxide nanoparticles were sonicated in 10 mL of methanol while adding 1 mL of oleic acid. After dispersion of the nanoparticles, the mixture was incubated with 2.5 mL of an aqueous solution of the selected ligand (see section 3.1) at 1%. If needed, sonication was applied to the mixture. The dispersed nanoparticles in aqueous phase were washed with water by centrifugation.

Oleate-capped nanoparticles can be also oxidized with sodium periodate in aqueous solution to produce the transfer to aqueous phase. Oleic acid is an unsaturated fatty acid with a double carbon bond at C9 position that can be cleaved by oxidation to produce the azelaic and pelargonic acids rendering the nanoparticles dispersed in aqueous media.

3.3. Preliminary Results for Functionalization of Magnesium Oxide Nanoparticles.

Magnesium oxide nanopowder was sonicated in the presence of different ligands: 2-aminoethylphosphonic acid, alendronate, carboxylated polyethylene glycol, dopamine and dihydroxyhydrocinnamic acid. 2-aminoethylphosphonic acid and cathecol-based ligands dissolved the nanomaterial. Catechol-based ligands also polymerized in the presence of magnesium oxide. FIG. 7 shows the dispersion yield of magnesium oxide nanomaterial with that gave better results.

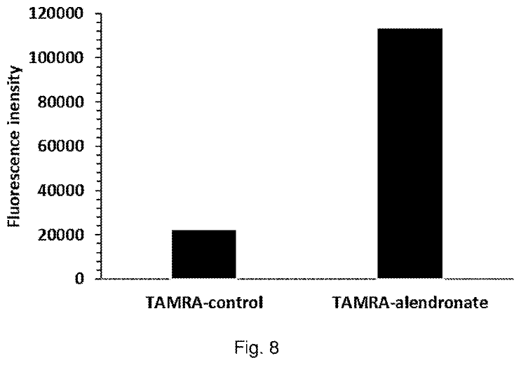

Given the good results with alendronate, a fluorescence probe synthesized by conjugating TAMRA fluorophore with alendronate was evaluated for the labelling of these nanomaterials. As it can be seen in FIG. 8, this fluorescence probe labels the nanoparticles. This property has important implications in biodistribution studies of the nanomaterial.

1.-20. (canceled)

21. A nanoparticle material comprising or consisting of a resorbable nanoparticle being functionalized with at least one compound,
the at least one compound comprising a capping agent and an agent capable of binding to a pathogen,
the capping agent being directly attached to a surface of the resorbable nanoparticle, and
said agent capable of binding to a pathogen being attached via said capping agent to the surface of said resorbable nanoparticle.

22. The nanoparticle material according to claim 21, wherein said resorbable nanoparticle comprises at least one metal.

23. The nanoparticle material according to claim 22, wherein said at least one metal is selected from the group consisting of magnesium, iron and zinc.

24. The nanoparticle material according to claim 21, wherein said resorbable nanoparticle comprises at least one metal salt.

25. The nanoparticle material according to claim 21, wherein said resorbable nanoparticle comprises:
a core comprising or consisting of at least one metal; and
a sheath comprising or consisting of at least one metal salt.

26. The nanoparticle material according to claim 21, wherein said resorbable nanoparticle has an average diameter of between 0.1 nm and 500 nm.

27. The nanoparticle material according to claim 21, wherein said resorbable nanoparticle has a shape selected from the group consisting of a cube shape, a cuboid shape, a prism shape, a tetrahedral shape, a cylindrical shape, a triangular shape, a pyramidal shape, a cone shape, an egg shape, a spherical shape, a star shape, a rod shape and combinations thereof.

28. The nanoparticle material according to claim 21, wherein said at least one compound is attached covalently and/or non-covalently to a surface of said resorbable nanoparticle.

29. The nanoparticle material according to claim 21, wherein said capping agent is selected from the group consisting of catecholates, salicylates, phosphates, phosphonates, bisphosphonates, polyethylene glycol conjugated with a catecholate, polyethylene glycol conjugated with a salicylate, polyethylene glycol conjugated with a phosphate, polyethylene glycol conjugated with a phosphonate, polyethylene glycol conjugated with a bisphosphonate, poly-glutamic acid conjugated with a catecholate, poly-glutamic acid conjugated with a salicylate, poly-glutamic acid conjugated with a phosphate, poly-glutamic acid conjugated with a phosphonate, poly-glutamic acid conjugated with a bisphosphonate, poly-aspartic acid conjugated with a catecholate, poly-aspartic acid conjugated with a salicylate, poly-aspartic acid conjugated with a phosphate, poly-aspartic acid conjugated with a phosphonate, poly-aspartic acid conjugated with a bisphosphonate and mixtures of at least two of the afore-mentioned capping agents.

30. The nanoparticle material according to claim 21, wherein said agent capable of binding to a pathogen is selected from the group consisting of an antibody directed against said pathogen, a protein, a polypeptide, an oligopeptide, a nucleic acid, a lipid, an antigen and mixtures thereof.

31. The nanoparticle material according to claim 21, wherein said resorbable nanoparticle comprises at least one metal oxide and/or at least one metal hydroxide.

32. The nanoparticle material according to claim 21, wherein said resorbable nanoparticle comprises at least one metal oxide selected from the group consisting of magnesium oxide, iron oxide, zinc oxide and mixtures thereof.

33. The nanoparticle material according to claim 21, wherein said resorbable nanoparticle comprises at least one metal hydroxide selected from the group consisting of magnesium hydroxide, iron hydroxide, zinc hydroxide and mixtures thereof.

34. A method for preventing and/or treating a disease and/or a disorder in a subject comprising the step of administering the nanoparticle material according to claim 21.

35. The method according to claim 34, wherein said disease and/or disorder is caused by a pathogen of fungal, viral or bacterial origin.

36. The method according to claim 34, wherein said disease and/or disorder is caused by a pathogen of viral origin, and wherein said pathogen of viral origin is SARS-CoV-2 or a virus of coronavirus type.

37. The method according to claim 34, wherein said disease and/or disorder is a proliferative disease.

38. The method according to claim 37, wherein said proliferative disease is a disease associated with some degree of abnormal cell proliferation, preferably cancer, wherein said cancer is preferably selected from the group consisting of carcinoma, lymphoma, blastoma, sarcoma, leukemia, non-localized cancer, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, skin cancer, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulvar cancer, thyroid cancer, hepatic carcinoma, head cancer and neck cancer.

39. A process for obtaining a nanoparticle material according to claim 21 comprising the steps of:
a) coating a resorbable nanoparticle with an antioxidant agent; and
b) replacing the coating of the antioxidant agent by functionalizing the resorbable nanoparticle with at least one compound being different from the antioxidant agent.

40. A product comprising the nanoparticle material according to claim 21, wherein the product is a pharmaceutical composition, a medical kit or a medical device.

41. A method of imaging, generating radicals, disinfecting surfaces or detecting a pathogen and/or an analyte comprising the step of using the nanoparticle material according to claim 21.
